(19) United States
(12) Patent Application Publication
(10) Pub. No.: US 2004/0088004 A1
(43) Pub. Date: May 6, 2004

(54) Suture anchor

(76) Inventor: Theodor Gerhard Rosch, Pretoria (ZA)

Correspondence Address: HARNESS, DICKEY & PIERCE, P.L.C., P.O. BOX 828, BLOOMFIELD HILLS, MI, 48303, US

(21) Appl. No.: 10/221,520

(22) PCT Filed: Mar. 9, 2001
(86) PCT No.: PCT/IB01/00335

(30) Foreign Application Priority Data
Mar. 13, 2000 (ZA) 2000/1282

(51) Int. Cl. A61B 17/04

(52) U.S. Cl. 606/232

(57) ABSTRACT

A suture anchor is provided which includes an attachment formation for attaching a suture to the anchor, the attachment formation being configured to allow relative movement between the suture and the anchor in an operative direction and to inhibit relative movement between the suture and the anchor in an operative direction. Typically, the suture anchor includes two attachment formations arranged in side by side fashion. In certain embodiments, the attachment formation is defined by a passage that extends through the anchor, the passage being shaped and dimensioned to inhibit retraction of the suture in the operative direction. The attachment formation and the anchoring portion may be composite or integrally formed. The invention extends to a suture anchor attachment component for attaching a suture to a suture anchor and to a method of attaching tissue to an anchoring substrate. Further, the invention also extends to a suture anchor insertion tool.

THIS INVENTION relates to a suture anchor. It also relates to a suture anchor attachment component and to a method of attaching tissue to an anchoring substrate.

In orthopaedic surgery it is often necessary to attach tissue to bone with sutures. Conventionally, sutures were passed through holes made in the bone and the sutures were thereafter secured to the bone by means of knots. In addition to bone tunnelling techniques, suture anchors are also readily available for securing tissue to bone. The suture anchor may be anchored to the bane in various different ways, e.g. a bore may be drilled into the bone, and the suture is then tied or knotted to the anchor. The surgeon usually operates in a relatively confined area, particularly when operating through a cannula, making it difficult to tension and secure the suture to the suture anchor in a satisfactory fashion. For the purposes of this specification, the application of the invention in securing tissue to bone should be predominantly, but not exclusively, borne in mind. Further, the term suture should be interpreted broadly to include any flexible securing means such as stitches, or the like and, likewise, the term tissue should be interpreted broadly to include tendons, ligaments, muscles, prostheses, or the like.

According to the invention, there is provided a suture anchor which includes an attachment formation for attaching a suture to the anchor, the attachment formation being configured to allow relative movement between the suture and the anchor in an inoperative direction and to inhibit relative movement between the suture and the anchor in an operative direction.

During surgery, a surgeon can manipulate or displace the suture until it is satisfactorily located or configured, whereafter the suture may be urged into an operative condition of the suture anchor in which relative movement between the suture and the anchor in the operative direction is inhibited.

The anchor is typically used to attach tissue to bone. The anchor may be inserted longitudinally into a bore in the bone and a surgeon may attach the suture to the tissue and feed the suture through the attachment formation and draw the suture in the inoperative direction through the attachment formation thereby to pull the tissue towards the bone. As relative movement in the operative direction is inhibited, the suture secures the tissue to the bone without the need for any knots.

The suture anchor may include a plurality of attachment formations thereby to allow several sutures to be secured to the anchor. Typically, the suture anchor includes two attachment formations arranged in side by side fashion.

The attachment formation may be defined by a passage that extends through the anchor, the passage being configured, e.g. shaped and dimensioned, to inhibit retraction of the suture in the operative direction. In certain embodiments, the passage has a cross-section that tapers to define a narrow holding zone in which the suture is receivable with a friction fit, The passage may further include a broad release zone. When the suture is urged into the holding zone removal thereof may be opposed by friction. Typically, the cross-section of the passage is “V”-shaped in outline and the passage extends at an angle, e.g. transversely, relative to a longitudinal axis of the anchor.

The passage may be defined by side walls with holding formations, e.g. grooves or the like, to assist in holding the suture in the passage. The holding formations are preferably configured to encourage movement of the suture into a captive zone of the passage. The holding formations are also preferably configured to hold sutures of different thickness. Accordingly, the size of the passage may depend on the thickness or size of the suture which it configured to hold in use, the size of the anchor, its intended application, or the like.

The attachment formation may be configured substantially to resemble a cleat.

The suture anchor may include an anchoring portion for anchoring the anchor to a base, the anchoring portion being attached in use to the attachment formation. The anchoring portion may be selected from the group consisting of an expanding type, screw-type, umbrella-type and wedge-type arrangement, or the like.

In certain embodiments, the anchoring portion is integrally formed with the attachment formation. In other embodiments, the attachment formation and the anchoring portion are composite including complemental connection means thereby to interconnect the attachment formation and the anchoring portion.

The attachment portion may be cylindrical in shape and one or more passages may extend laterally through the attachment portion. Typically, the relative size of the anchor and the bore provided in the bone is such that the passage is located within the bone in use. Accordingly, in order to facilitate movement or travel of the suture through the passage during the anchoring process, the anchor may include guide means for guiding movement of the suture through the holding formation. The guide means may be in the form of a pair of shoulders provided at opposed sides of the holding formation.

Further in accordance with the invention, there is provided a suture anchor, which includes an attachment formation for attaching a suture to the anchor, the attachment formation including holding means configured to hold the suture captive by means of friction.

The suture anchor may thus function as a so-called “knotless” suture anchor. The anchor and its various components may be manufactured from any suitable material, absorbable or not absorbable.

The invention extends to a suture anchor attachment component for attaching a suture to a suture anchor, the component including

an attachment formation configured to allow relative movement between the suture and the anchor in an inoperative direction and to inhibit relative movement between the suture and the anchor in an operative direction; and

connection means for connecting the suture anchor attachment component to the suture anchor, the suture anchor including complemental connection means.

Still further in accordance with the invention, there is provided a method of attaching tissue to an anchoring substrate with a suture anchor which includes an attachment formation which allows relative movement between a suture and the anchor in an inoperative direction and inhibits relative movement between the suture and the anchor in an operative direction, the method including

securing at least one suture to the tissue to define a secured end of the suture;

inserting free ends of the suture into the attachment formation in the inoperative direction;

locating the anchor into the anchoring substrate; and

drawing the suture through the attachment formation thereby to draw the tissue towards the anchoring substrate.

The method may be performed in arthroscopic applications using at least one-cannula. In certain circumstances, the method includes attaching the suture to the tissue with arthroscopic suture means, e.g. a suture punch or the like, and withdrawing the free ends of the suture through the cannula. The free ends may be drawn through the attachment formation and, whilst a surgeon is holding the free ends, the anchor may be slid along the suture through the cannula towards the anchoring substrate.

The suture may be drawn through the attachment formation until the tissue is in at least close proximity to, preferably, snugly located against, the anchoring substrate in the form of bone and thereafter urging the suture in the operative direction to enhance the grip of the attachment formation on the suture. The bore may be shaped to facilitate movement of the suture through the attachment formation when the anchor is located within the bore.

For example, the tissue may be rotator cuff muscle and the anchoring substrate may be bone of the shoulder joint. It is however to be appreciated that the term “tissue” may include soft tissue, tendons, or the like which require attachment.

The anchor may be located in a pre-drilled bore in the bone and secured therein in a conventional fashion. Further, conventional instrumentation may be used to execute one or more steps of the invention.

In certain circumstances, the bore may be shaped or broached to facilitate movement or travel of the suture through the attachment formation when the anchor is located within the bore.

Still further in accordance with the invention, there is provided a suture anchor insertion tool for inserting into a bore a suture anchor including an attachment formation for attaching a suture to the anchor, the attachment formation defining a captive zone which is configured to allow relative movement between the suture and the anchor in an inoperative direction and to inhibit relative movement between the suture and the anchor in an operative direction and the tool including

holding means for holding the suture anchor; and

checking means for checking movement of the suture into the captive zone to allow movement in both the operative and inoperative directions.

The suture anchor insertion tool may include

a handle portion to facilitate use of the tool by a user; and

an elongate shaft extending at its fixed end from the handle portion and terminating at a free end portion which defines a hollow holding zone in which the suture anchor is at least partially located in use, the checking means being configured to check displacement of the suture, into the captive position when the suture anchor is located in the free end portion.

The free end portion may define a hollow cylindrical rim operatively forming a checking surface against which the suture abuts in use.

The invention is now described, by way of example, with reference to the accompanying diagrammatic drawings.

In the drawings,

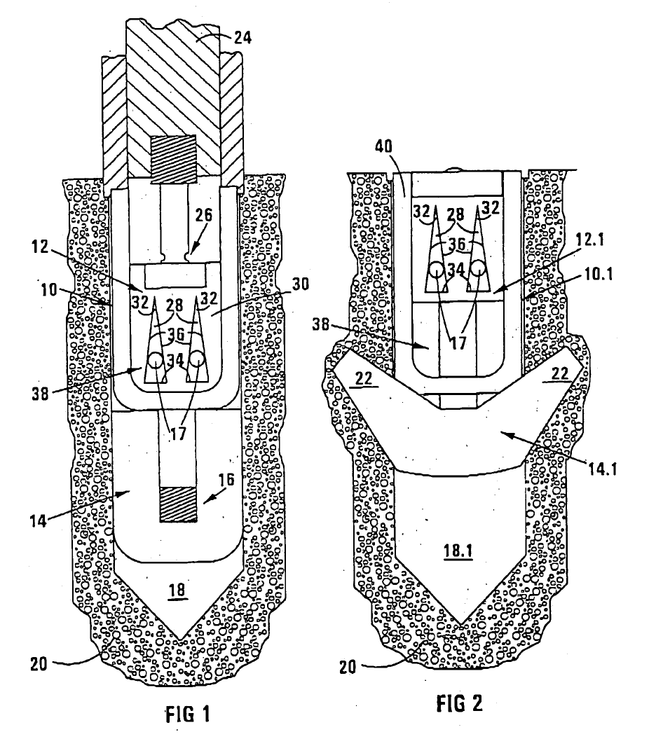

FIG. 1 shows a partly sectional view of a suture anchor, in accordance with the invention, inserted in a bore formed in a bone;

FIG. 2 shows a partly sectional view of a further embodiment of a suture anchor, in accordance with the invention, in its anchored condition using an expanding-type anchor as an example;

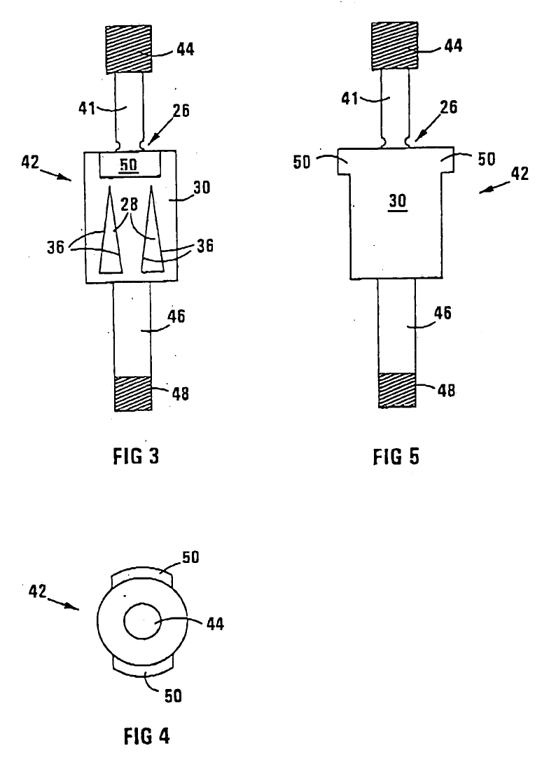

FIG. 3 shows a front view of an attachment component, also in accordance with the invention, of the suture anchor of FIG. 1;

FIG. 4 shows a top plan view of the attachment component of FIG. 3;

FIG. 5 shows a side view of the attachment component of FIG. 3;

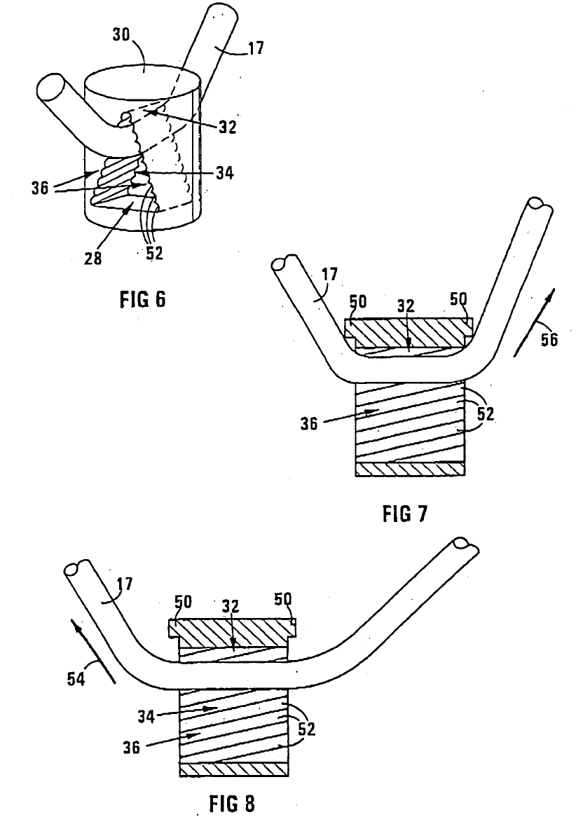

FIG. 6 shows an example of an attachment or holding formation of the attachment component of FIGS. 3 to 5;

FIGS. 7 and 8 show longitudinal sectional views of the attachment formation;

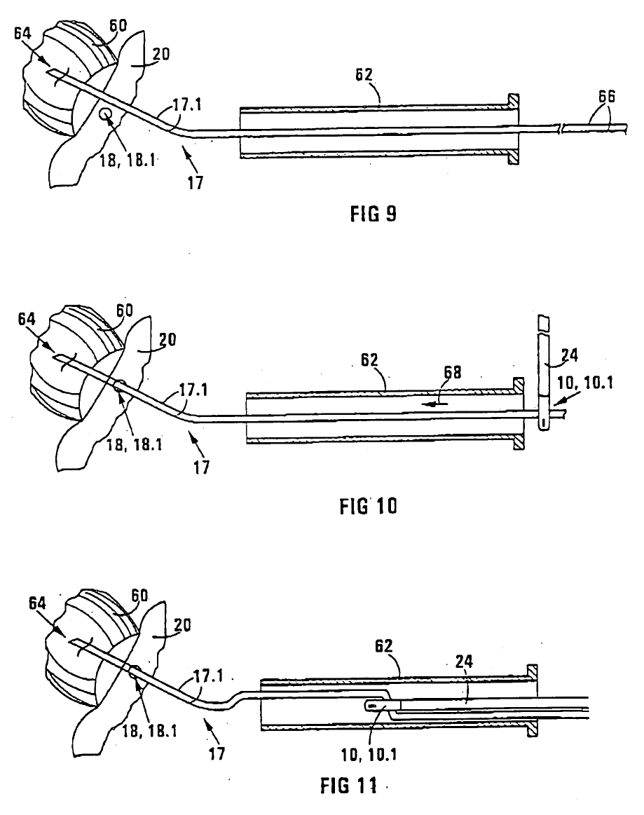

FIGS. 9 to 13 show a method of attaching tissue to an anchoring substrate in the form of bone;

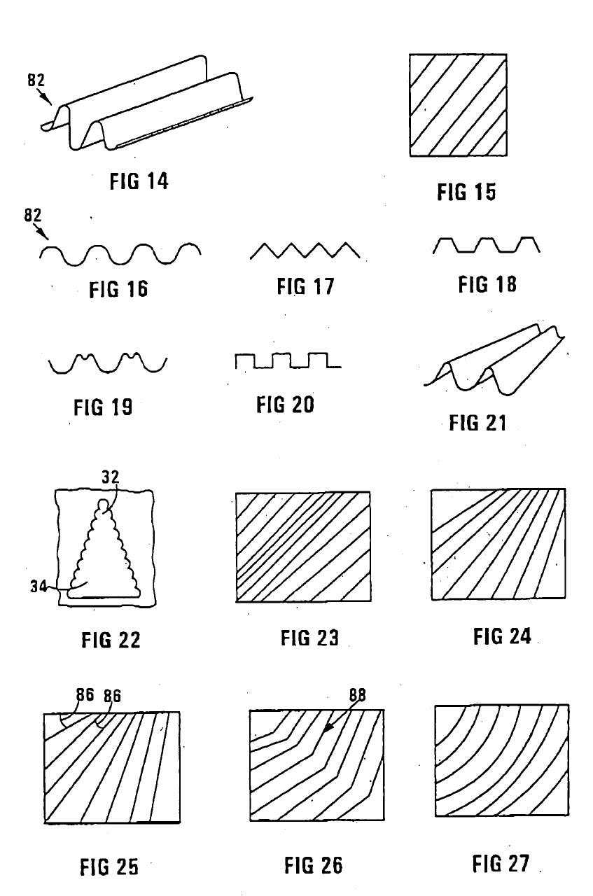

FIGS. 14 to 28 show examples of different shapes and configurations of attachment or holding formations of the suture anchor;

FIGS. 29 and 30 show schematic representations of curved upper walls of the holding formations to facilitate limited travel of a suture through the formation;

FIGS. 31 to 34 show examples of different shapes which lateral passages of the suture anchor may have when viewed in cross-section;

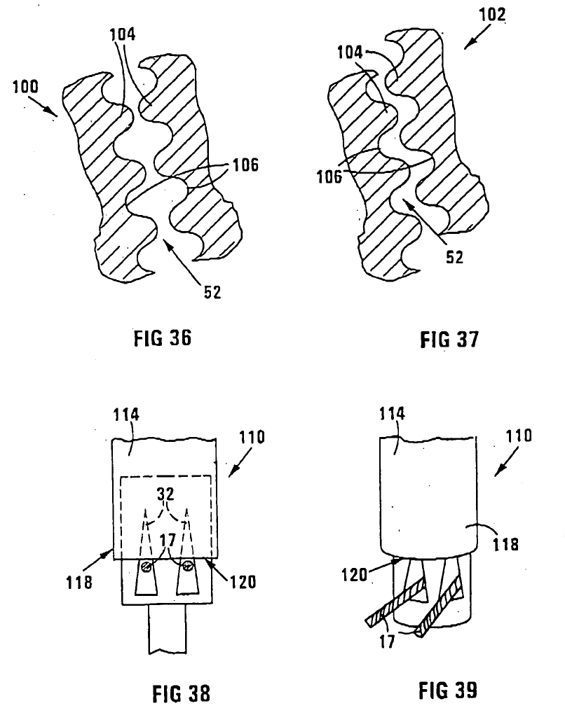

FIG. 35 shows a schematic representation of a top view of a further embodiment of a suture anchor in accordance with the invention; 125 FIGS. 36 and 37 show cross-sectional views of further embodiments of holding formations of the suture anchor;

FIG. 38 shows a schematic representation of a front view of a distal or terminal end of a suture anchor insertion tool, also in accordance with the invention;

FIG. 39 shows a three-dimensional view of the distal end of the tool of FIG. 38;

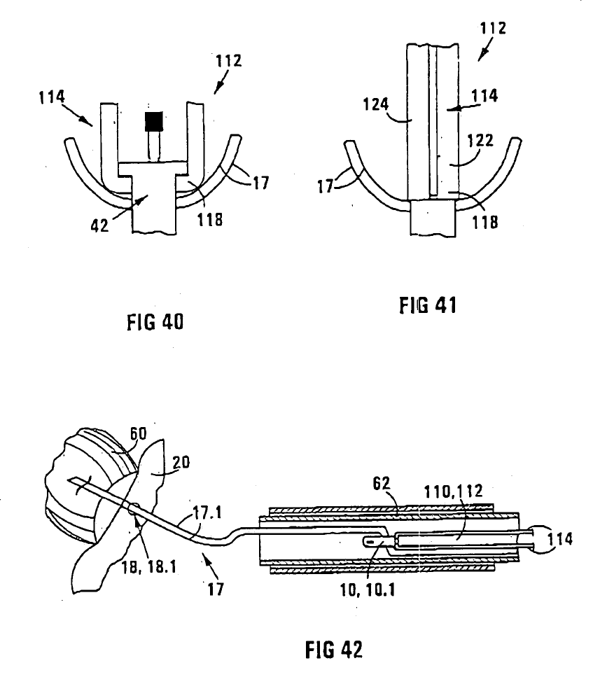

FIG. 40 shows a side view of a further embodiment of a suture anchor insertion tool in accordance with the invention;

FIG. 41 shows a front view of the tool of FIG. 40;

FIG. 42 shows the suture anchor tool of FIGS. 40 and 41 in use;

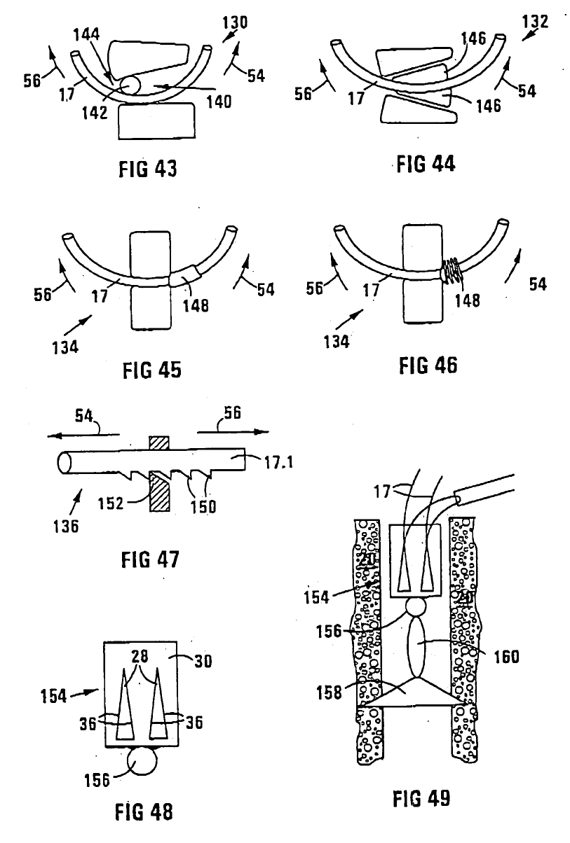

FIGS. 43 to 47 generally indicate further embodiments of holding formations, also in accordance with the invention;

FIG. 48 shows a schematic front view of a further embodiment of an attachment component, also in accordance with the invention, for anchoring a suture in bone; and

FIG. 49 shows a schematic representation of the attachment component of FIG. 48.

Referring in particular to FIG. 1 of the drawings, reference numeral 10 generally indicates a suture anchor in accordance with the invention. The suture anchor 10 includes an attachment portion or component 12 and an anchoring portion or component 14 which are secured or connected to each other by a complementary screw-thread arrangement 16. As described in more detail below, the suture anchor 10 is typically used to attach or secure tissue to bone. For example, the suture anchor 10 may be used to attach a rotator cuff muscle to bone of a shoulder joint by means of a suture 17.

The anchoring component 14 may be any conventional arrangement such as an expanding-type, a screw-type, an umbrella-type, a wedge-type, or the like arrangement used in conventional surgery. As shown in FIG. 1, the anchor 10 may be inserted into a bore 18 drilled into bone 20. A conventional insertion instrument 24 may be used by the surgeon to insert the anchor 10 and, accordingly, the anchor 10 may include a conventional snap arrangement 26 to facilitate removal of the instrument 24. Of particular importance is that the attachment component 12 includes an attachment or holding formation which, in this embodiment is defined by two spaced lateral passages 26 which extend laterally through a body portion 30 of the attachment component 12. The lateral passages 28 are triangular or “V” shaped when viewed in cross-section defining a narrow holding tone 32 and a broad release zone 34. As can be clearly seen in the drawings, side walls 36 of the passages 28 taper from the release zone 34 towards the holding zone 32.

Referring in particular to FIG. 2 of the drawings, reference numeral 10.1 generally indicates a further embodiment of a suture anchor in accordance with the invention. The suture anchor 10.1 substantially resembles the suture anchor 10 and, accordingly, like reference numerals have been used to indicate the same or similar features unless otherwise indicated. In the suture anchor 10, the passages 28 are located towards a lower region 38 of the attachment component 12. However, in the suture anchor 10.1, the passages 28 are located towards a superior or upper region 40 of the attachment component 12.1. Further, the suture anchor 10 is composite in nature and its attachment component 12 forms an independent component which is attached to a conventional anchoring component. However, in the embodiment of the invention depicted in FIG. 2 of the drawings, the attachment component 12.1 is integrally formed with the anchoring component 14.1. In use, when the suture anchor 10.1 is inserted into its associated bore 18.1, projections 22 penetrate the bone 20 to retain the anchoring component 14.1 within the bore 18.1 to inhibit withdrawal thereof from the bore 18.1.

Referring in particular to FIGS. 3 to 5 of the drawings, reference numeral 42 generally indicates a further embodiment of an attachment component, also in accordance with the invention, for attachment to a conventional or otherwise anchoring component. The attachment component 42 substantially resembles the attachment component 12 and, accordingly; like reference numerals have been used to indicate the same or similar features unless otherwise indicated.

The attachment component 42 includes a connecting member 41 with connection means 44 for connecting the attachment component 42 to an insertion tool (not shown) which may be substantially similar to the insertion tool 24, A further connecting member 46 is provided with a screw threaded end portion 48 for mounting the attachment component to an anchoring component (not shown) which may be substantially similar to the anchoring component 14 (see FIG. 1). A body portion 30 of the attachment component 42 is cylindrical in outline and has guide formations in the form of rectangular abutments or shoulders 50 which guide travel of a suture (not shown), as described in more detail below with reference to FIGS. 7 and 8.

The side walls 36 have holding formations in the form of grooves 52 (only a few of which are referenced in the drawings) to assist in holding the suture 17 in the narrow captive or holding zone 32 of the passage 28. The grooves 52 are orientated so that the passage 28 with its side walls 3C forms a cleat which holds the suture 17 captive within the passage 28 in a substantially similar fashion to a conventional cleat which holds a rope, for example, on a sail boat or the like. In particular, the passage 28 with its grooves 52 defines a holding formation or captive zone in which relative movement or travel between the suture 17 and the attachment component 42 (and thus the suture anchor 10) is allowable in an inoperative direction 54 (see FIG. 8). However, relative movement between the suture 17 and the attachment component 42 (and thus the suture anchor 10) in an operative direction 56 is inhibited (see FIG. 7). Thus, the operative direction 56 defines a direction in which displacement or movement of the suture 17 is inhibited and the suture is thus held captive or “snagged”, Accordingly, unlike conventional suture anchors, no knot is required to retain the suture in its holding condition. The inoperative direction 54 defines a direction in which movement or displacement of the suture 17 is permissible by a surgeon. The suture anchor 10 thus defines an operative condition in which the suture 17 is held captive in the attachment component 42 and an inoperative condition in which displacement of the suture 17 in the inoperative direction 54 is permissible. The suture anchor 10, 10.1 allows retensioning of a suture if necessary; For example, if during surgery the tension on tissue or cuff is initially high and subsequently decreases, the surgeon may then increase the tension by drawing the suture 17 in the operative direction 56. Likewise, if the tension is too high, the surgeon may remove the suture 17 from its captive position in the holding zone 32 (see FIGS. 1, 2, 6, 7, and 8) and displace it into the release zone 34 in which free movement of the suture 17 is permissible. Thus, adjustment and re-adjustment of the tension in the suture 17 is permissible by the surgeon. In certain circumstances in which the surgeon no longer, requires the suture, it may be totally removed by drawing the suture; 17 in the inoperative direction 54 until it is free of the suture anchor 10.

In a similar fashion to a cleat, and due to the grooves 52, when the suture 17 is drawn in the operative direction 56 the suture 17 is urged by the grooves 52 towards the narrow holding zone 32. However, due to the orientation of the grooves 52, when the suture 17 is urged in the inoperative direction 54 the grooves 52 encourage displacement of the suture 17 away from the narrow holding zone 32 towards the release zone 34. In order to facilitate displacement of the suture 17 away from the narrow holding zone 32, the rectangular abutments 50 are provided. The rectangular abutments 50 assist in preventing the suture 17 from being frictionally retained within the narrow holding zone 32 to such an extent that the suture 17 cannot be drawn in the inoperative direction 54. Due to the arrangement of the abutments 50, even when the suture anchor 10, 10.1 is located within the bore 18, 18.1, displacement of the suture 17 in the inoperative direction 54 is permissible thereby enabling the surgeon to draw the tissue towards the bone 20 as described in more detail below. In certain embodiments as shown in FIGS. 29 and 30, curved upper walls 80 are provided at an upper end of the passage 28 to facilitate travel of the suture in the inoperative direction.

Referring in particular to FIGS. 9 to 13, a method of attaching tissue in the form of a rotator cuff muscle 60 to bone 20 of a shoulder joint is illustrated. The method may be performed by means of endosurgery using a cannula 62. Rotator cuff muscles 60 are often torn from the bone 20 and, accordingly, may thus require re-attachment thereto by means of the suture 17.

Initially, the surgeon drills a bore 18 by means of conventional techniques in the humeral head of the shoulder joint. Thereafter, strands 17.1 of a conventional suture 17 are attached at their secured end 64 to the rotator cuff muscle 60 by any conventional means. For example, the secured ends 64 may be attached by means of a suture punch or the like. The suture 17 is then arranged so that it extends through the cannula 62 with its free ends 66 exposed (see FIG. 9). Thereafter, the free ends 66 of the suture 17 are each fed through a passage 23 of the suture anchor 10, 10.1 in the inoperative direction 54 (see FIGS. 1, 2, 8 and 10). Thereafter, the surgeon grips the free ends 66 and displaces or urges the suture anchor 10, 10.1 along the suture 17 in the direction of arrow 68 by means of the insertion tool 24 (see FIG. 11). During this process, the suture anchor 10, 10.1 is displaced relative to the suture 17 in the inoperative direction 54 and, accordingly, the suture 17 is not held captive in the narrow holding zone 32.

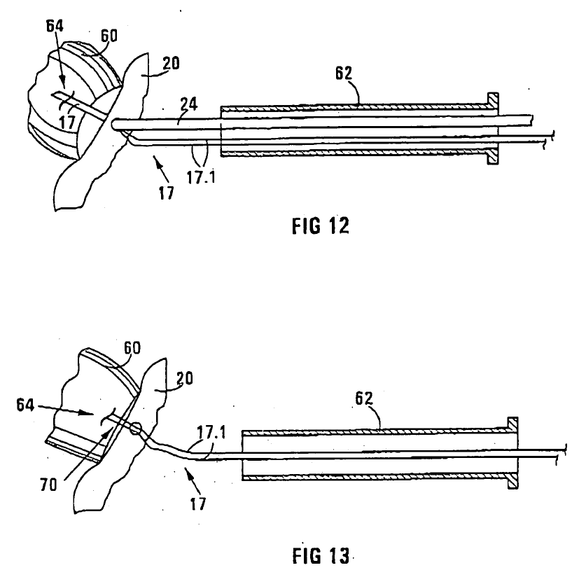

Once the suture anchor 10, 10.1 has been displaced almost along the entire length of the suture 17 by means of the insertion tool or instrument 24, the surgeon locates the suture anchor 10, 10.1 in the bore 18, 18.1 (see FIG. 12) and activates the anchoring component 14, 14.1 of the suture anchor 10, 10.1, in a conventional fashion. The insertion tool is then removed. Thereafter, the surgeon draws the suture 17 through the attachment component 12, 12.1 thereby drawing the rotator cuff muscle 60 towards the bone 20 as shown in FIG. 13. Due to the configuration of the attachment formation 12, the suture 17 cannot be withdrawn in the operative direction 56 and, accordingly, the rotator cuff muscle 60 is held and attached to the bone 20. In certain circumstances, the surgeon may then, as a safety feature, knot the strands 17.1 in a conventional fashion whereafter they are trimmed to an appropriate length. It is to be appreciated that the method may be performed to attach any tissue to a substrate.

After the surgeon has drawn the rotator cuff muscle 60 into its desired position relative to the bone 20, force may be applied on a connecting portion 70 of the suture 17 so that the suture 17 is drawn snugly into the narrow holding zone 32.

It is to be appreciated that the shape and dimensions of the suture anchor 10, 10.1 may vary for different applications. For example, when viewed in top plan the suture anchor 10, 10.1 may, for example, in outline be round, square, hexagonal, oval, or the like. In certain embodiments, the suture anchor may include tapered protrusions 71, e.g. fins or the like, which may upon insertion cut into walls of the bone in which the bore has been formed (see FIG. 35). Further, it is to be appreciated that the size and/or shape of the lateral passages 28 may also vary depending on the type of suture used.

Referring in particular to FIGS. 14 to 28 of the drawings, various examples of different shapes and configurations of holding formations of the suture anchors 10, 10.1 are illustrated. FIG. 14 shows wave-like formations 82 which are orientated to retain the suture 17 in the holding formations. FIG. 16 shows a cross-sectional view of the wave-like formations 82 and FIGS. 17 to 20 show further examples of holding formations. It is to be appreciated that the holding formations may be of any other suitable shape and/or orientation for restricting movement or travel of the suture 17 in an operative direction.

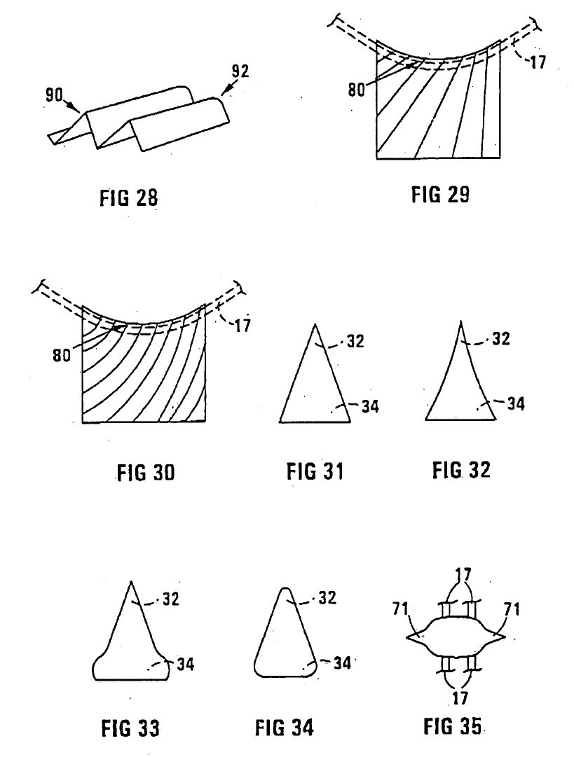

In the same way as the holding formations may differ in size end shape, they may also differ in orientation and longitudinal form. For example, the holding formations may be spaced equidistantly, (see FIG. 15), or at different angles, the formations may vary longitudinally in height (see FIG. 21), the shape and configuration of the formations may vary from the release zone 34 towards the holding zone 32 (see FIG. 22), or the distance or spacing between the formations may vary (see FIGS. 23 and 24). Further, the relative angles 86. (see FIG. 25) between the formations may vary, the pitch of the formations may vary (see FIG. 26), the formations may be curved (see FIG. 27), the formations may gradually change from a sharp cross-sectional profile 90 to a rounded cross-sectional profile 92 (see FIG. 28) or the like. It is to be appreciated that the formations may be a combination of the aforementioned and can be of any other form suitable to retard or inhibit displacement of the suture in one direction.

Referring to FIGS. 36 and 37 of the drawings, reference numerals 100, 102 show further embodiments of holding formations of a suture anchor In accordance with the invention. The holding formations 100, 102 both include grooves 52 or wave-like formations as described above. The formations 100 are arranged so that opposed peaks 104 and troughs 106 are aligned whereas, in the case of the holding formation 102, the peaks 104 of one side wall are aligned with the troughs 106 of the other opposed side wall. The configurations of the holding formations depend, on the type of material from which they are manufactured, the type of suture with which it is to be used (e.g. monofilament, multifilament, soft, hard, or the like sutures), or the specific application of the suture anchor. Further, the holding formations with their optional grooves 52, may have various different surface finishes. For example, the surfaces may be roughened by bead blasting to increase the friction between the suture and the holding formation, the surfaces may have indentations resembling those provided on a golf ball, raised protrusions may be provided on the surfaces, or the like.

Referring to FIGS. 38 to 41 of the drawings, reference numerals 110 and 112 generally indicate a suture anchor insertion tool, also in accordance with the invention. The suture anchor insertion tools 110, 112 include a handle portion (not shown) which the surgeon grips in use and which may resemble the handle of a conventional suture anchor insertion tool, and an elongate shaft 114. The shaft 114 extends from its fixed end where it is attached to the handle portion and terminates at a free end portion 118 which defines a hollow holding zone which a suture anchor, for example the suture anchor 10, 10.1, is received in use. In particular, the attachment component 42 (see FIG. 3) of the suture anchor 10, 10.1 is typically at least partially received within the free end portion 118. The free end portion 118 of the insertion tool 110 defines a circular rim forming a checking surface 120 against which the suture 17 may abut in use. In particular, the checking surface 120 prevents the sutures 17 from entering she holding zone 32 and hereby allowing relative movement between the suture 17 and the anchor 10, 10.1. Upon removal of the insertion tool 110, 112 the suture 17 may then be moved or displaced into its captive position in which it is held by means of a friction fit in the holding zone 32. The suture anchor insertion tool 112 is substantially similar to the suture anchor insertion tool 110 and, accordingly, functions in a substantially similar way. However, unlike the tool 110 which has a hollow circular cylindrical continuous side wall, the tool 112 has a composite elongate shaft comprising two elongate components 122, 124 which also define a hollows circular cylindrical opening for receiving the suture anchor 10, 10.1. However, in order to remove the insertion tool 112 the components 122, 124 are displaced away from each other thereby to release the suture anchor 10, 10.1 from the tool 112.

Thus, the insertion tools 110, 112 are configured in such a fashion so as to prevent displacement or movement of the suture 17 into its captive zone or holding formation in which movement is restricted in the operative direction and, accordingly, the suture anchor 10, 10.1 may be slid in any direction along the suture 17. Once the suture anchor 10, 10.1 has been located almost along the entire length of the suture 17 by means of the insertion tool 110, 112 the surgeon may then locate the suture anchor 10, 10.1 in a bore in a bone and activate the bone anchoring component 14, 14.1 of the suture anchor 10, 10.1 respectively in a conventional fashion. Upon withdrawal of the insertion tool 110, 112 the sutures 17 are no longer checked or inhibited from entering a captive position in the holding zone 32 and, accordingly, the surgeon may then urge the suture 17 into the holding zone 32 thereby to allow relative movement between the suture 17 and the suture anchor 10, 10.1 in an operative direction and to inhibit relative movement between the suture 17 and the anchor 10, 10.1 in the operative direction. The surgeon may then draw the rotator cuff muscle 60 towards the bone 20 as shown in FIG. 13 of the drawings. In certain circumstances, the surgeon may make multiple suture loops through the tissue, e.g. using the so-called “Mason-Allen” technique.

It is believed that an advantage of the insertion tool 110, 112 is that the suture anchor 10, 10.1 is gripped and may thus be withdrawn from, the bore if the surgeon so wishes. Further, in the event of the suture anchor being deployed in the bone 20, the insertion tool 110, 112 may be located about the suture anchor 10, 10.1 to dislodge the suture 17 from the holding zone 32 thereby to allow relative movement of the suture 17 in both the operative and inoperative directions. FIG. 42 shows a schematic representation of the tool 110, 112 in use.

Referring in particular to FIGS. 43 to 47 of the drawings, further embodiments of attachment portions or components are generally indicated by reference numerals 130, 132, 134, and 136. As is the case with the attachment components 12, 12.1, the attachment components 130 to 136 are configured so as to allow movement of a suture 17 in an inoperative direction 54 and restrict or inhibit movement or displacement of the suture 17 in en operative direction 56. However, unlike the attachment component 12 with includes no moving parts and holds the suture 17 captive by means of friction, the attachment components 130, 132, 134, and 136 include retaining members to inhibit displacement of the suture 17 in the operative direction. In particular, the attachment component 132 defines a tapered passage 140 (see FIG. 43) in which a ball or roller 142 is located. In use, when the suture 17 is drawn in the operative direction 56, the ball is displaced into a narrow end 144 of the passage 140 where it restricts further displacement of the suture 17 in the operative direction 66. However, in the event of the suture 17 being drawn in the inoperative direction 54, the ball 142 is dislodged from the narrow end 144 thereby allowing displacement or movement of the suture 17 in the inoperative direction 54. The same capturing or holding principle is applied in the attachment component 132 except that, instead of the ball 142, two wedge-like members 146 restrict or inhibit displacement of the suture 17 in the operative direction 56. In the attachment component 134 an outer sleeve or tube 148 of deformable material is provided. As shown in FIG. 46, upon displacement of the suture 17 the sleeve 148 is compressed or deformed thereby frictionally to engage the suture 17 and inhibit displacement of the suture 17 in the operative direction 56. It is however to be appreciated that various different configurations may be conceived in order to inhibit or prevent displacement of the suture 17 in the operative direction 56 yet allow selective displacement of the suture 17 in the inoperative direction 54, For example, the attachment component 136 (see FIG. 47) includes a modified suture 17.1 which includes shaped protrusions 150 which, in combination with a wedge-shaped formation 152 of the attachment component 136, restricts movement of the suture 17.1 in the operative direction 56.

Referring to FIGS. 48 and 49 of the drawings, reference numeral 154 generally indicates yet a further embodiment of an attachment component in accordance with the invention. The attachment component 154 is similar to the attachment component 42 and, accordingly, like reference numerals have been used to indicate the same or similar features unless otherwise indicated. However, unlike the attachment component 42 which has a connecting member 46 for mounting the attachment component 42 to an anchoring component in a rigid fashion, the attachment component 154 includes a connecting member 156 in the form of a loop or ring for connecting the attachment component 154 to an anchoring component 158 (see FIG. 49) in a flexible fashion. Typically, the attachment component 154 is connected via a suture loop 160 to the anchoring component 158 which, typically, is a PANALOCK™-type anchor. It is thus to be appreciated that the attachment component 42, 130 to 136 which selectively holds the suture captive and inhibits displacement or movement of the suture 17 in the operative direction 56 may be formed integrally with the suture anchor or as a separate component which is attached to a conventional or otherwise suture anchor.

The inventor believes that the invention, as illustrated, provides, an enhanced suture anchor 10, 10.1 which facilitates attachment of tissue such as muscle 60 to an anchoring substrate in the form of a bone 20 in that no knots are required to secure the suture 17 to the suture anchor 10, 10.1. Further; attachment of a muscle via a plurality of sutures to a bone 20 may be facilitated in that the sutures 17 may be sequentially drawn towards the bone thereby sequentially drawing the muscle 60 in a step-wise process towards the bone 20. For example, a first suture may be drawn to draw the muscle 60 towards the bone 20 a portion of the total distance, thereafter, a second suture may be drawn to draw a different portion of the muscle towards the bone 20, and so on. The process may be repeated several times, thereby slowly to draw the muscle towards the bore 20.

1. A suture anchor which includes an attachment formation for attaching a suture to the anchor, the attachment formation being configured to allow relative movement between the suture and the anchor in an inoperative direction and to inhibit relative movement between the suture and the anchor in an operative direction.

2. A suture anchor as claimed in claim 1, which includes a plurality of attachment formations thereby to allow several sutures to be secured to the anchor.

3. A suture anchor as claimed in claim 2, which includes two attachment formations arranged in side by side fashion.

4. A suture anchor as claimed in any one of the preceding claims, in which the attachment formation is defined by a passage that extends through the anchor, the passage being shaped and dimensioned to inhibit retraction of the suture in the operative direction.

5. A suture anchor as claimed in claim 4, in which the passage has a cross-section that tapers to define a narrow holding zone in which the suture is receivable with a friction fit.

6. A suture anchor as claimed in claim 5, in which the cross-section of the passage is “V”-shaped in outline and the passage extends at an angle relative to a longitudinal axis of the anchor.

7. A suture anchor as claimed in any one of the preceding claims 4 to 6 inclusive, in which the passage is defined by side walls with holding formations to assist In holding the suture in the passage.

8. A suture anchor as claimed in claim 7, in which the holding formations are configured to encourage movement of the suture into a captive zone of the passage.

9. A suture anchor as claimed in claim 8, in which the holding formations are configured to hold sutures of different thickness.

10. A suture anchor as claimed in any one of the preceding claims, in which the attachment formation is configured substantially to resemble a cleat.

11. A suture anchor as claimed in any one of the preceding claims, which includes an anchoring portion for anchoring the anchor to a base, the anchoring portion being attached in use to the attachment formation.

12. A suture anchor as claimed in claim 11, in which the anchoring portion is selected from the group consisting of an expanding type, screw-type, umbrella-type and wedge-type arrangement.

13. A suture anchor as claimed in claim 11 or claim 12, in which the anchoring portion is integrally formed with the attachment formation.

14. A suture anchor as claimed in claim 11 or claim 12, in which the attachment formation and the anchoring portion are composite including complemental connection means thereby to interconnect the attachment formation and the anchoring portion.

15. A suture anchor as claimed in any one of the preceding claims, in which the anchor includes guide means for guiding movement of the suture through the holding formation.

16. A suture anchor as claimed in claim 15, in which the guide means are in the form of a pair of shoulders provided at opposed sides of the holding formation.

17. A suture anchor, which includes an attachment formation for attaching a suture to the anchor, the attachment formation including holding means configured to hold the suture captive by means of friction.

18. A suture anchor attachment component for attaching a suture to a suture anchor, the component including
an attachment formation configured to allow relative movement between the suture and the anchor in an inoperative direction and to inhibit relative movement between the suture and the anchor in an operative direction; and
connection means for connecting the suture anchor attachment component to the suture anchor, the suture anchor including complemental connection means.

19. A method of attaching tissue to an anchoring substrate with a suture anchor which includes an attachment formation which allows relative movement between a suture and the anchor in an inoperative direction and inhibits relative movement between the suture and the anchor in an operative direction, the method including
securing at least one suture to the tissue to define a secured end of the suture;
inserting free ends of the suture into the attachment formation in the inoperative direction;
locating the anchor into the anchoring substrate; and
drawing the suture through the attachment formation thereby to draw the tissue towards the anchoring substrate.

20. A method as claimed in claim 19, which is performed in arthroscopic applications using at least one cannula.

21. A method as claimed in claim 20, in which the method includes attaching the suture to the tissue with arthroscopic suture means and withdrawing the free ends of the suture through the cannula.

22. A method as claimed in claim 21, in which the free ends are drawn through the attachment formation and, whilst a surgeon is holding the free ends, the anchor is slid along the suture through the cannula towards the anchoring substrate.

23. A method as claimed in claim 22, in which the suture is drawn through the attachment formation until the tissue is in at least close proximity to the anchoring substrate in the form of bone and thereafter urging the suture in the operative direction to enhance the grip of the attachment formation on the suture.

24. A method as claimed in any one of the preceding claims 19 to 23 inclusive, in which the bore is shaped to facilitate movement of the suture through the attachment formation when the anchor is located within the bore.

25. A suture anchor insertion tool for inserting into a bore a suture anchor including an attachment formation for attaching a suture to the anchor, the attachment formation defining a holding or captive zone which is configured to allow relative movement between the suture and the anchor in an inoperative direction and to inhibit relative movement between the suture and the anchor in an operative direction and the tool including
holding means for holding the suture anchor; and
checking means for checking movement of the suture into the captive zone to allow movement in both the operative and inoperative directions.

26. A suture anchor insertion tool as claimed in claim 25, which includes
a handle portion to facilitate use of the tool by a user; and
an elongate shaft extending at its fixed end from the handle portion and terminating at a free end portion which defines a hollow holding zone in which the suture anchor is at least partially located in use, the checking means being configured to check displacement of the suture into the captive position when the suture anchor is located in the free end portion.

27. A suture anchor insertion tool as claimed in claim 26, in which the free end portion defines a hollow cylindrical rim operatively forming a checking surface against which the suture abuts in use.

28. A new suture anchor, substantially as herein described and illustrated.

29. A new suture anchor attachment component, substantially as herein described and illustrated.

30. A new method of attaching tissue to an anchoring substrate, substantially as herein described and illustrated.

31. A new suture anchor insertion tool, substantially as herein described and illustrated.
